(19) United States
(12) Patent Application Publication
(10) Pub. No.: US 2026/0294511 A1
(43) Pub. Date: Oct. 1, 2026

(54) TECHNOLOGIES FOR OPERATING AN ENERGY-BASED SURGICAL INSTRUMENT BASED ON TIME-DEPENDENT CLAMP PRESSURE

(71) Applicant: Cilag GmbH International, Zug (CH)

(72) Inventors: Frederick E. Shelton, IV, Hillsboro, OH (US); Jason L. Harris, Lebanon, OH (US); Jeffrey D. Messerly, Cincinnati, OH (US)

(21) Appl. No.: 19/343,914

(22) Filed: Sep. 29, 2025

Related U.S. Application Data
(60) Provisional application No. 63/780,373, filed on Mar. 30, 2025.

(51) Int. Cl. A61B 18/14; A61B 90/00; A61B 18/00

(52) U.S. Cl. CPC A61B 18/1445; A61B 90/06; A61B 2018/00601; A61B 2018/00678; A61B 2018/00702; A61B 2018/00714; A61B 2018/00779; A61B 2018/00791; A61B 2018/00982; A61B 2018/00994; A61B 2090/064

(57) ABSTRACT

Technologies for operating an energy-based surgical instrument based on a time-dependent clamp pressure includes controlling a jaw assembly of the surgical instrument to apply a pre-energy mode clamp pressure to patient tissue captured in the jaw assembly prior to application of a requested energy mode of the surgical instrument, activating the requested energy mode subsequent to application of the pre-energy mode clamp pressure to the patient tissue, and controlling the jaw assembly to apply an energy mode clamp pressure to the patient tissue while the requested energy mode is activated. Additionally, the jaw assembly may be controlled to apply a pre-tissue transection clamp pressure to patient tissue prior to transection of the patient tissue. In some embodiments, the temperature of the patient tissue may be increased prior to application of the requested energy mode.

### CROSS-REFERENCE TO RELATED APPLICATIONS

This application claims the benefit of and priority to U.S. Patent Application No. 63/780,373, entitled “TECHNOLOGIES FOR OPERATING AN ENERGY-BASED SURGICAL INSTRUMENT BASED ON TIME-DEPENDENT CLAMP PRESSURE,” which was filed on Mar. 30, 2025, and which is incorporated herein by reference in its entirety.

### TECHNICAL FIELD

The present disclosure relates generally to energy-based surgical instruments and, more particularly, to harmonic and/or electrosurgical surgical instruments.

### BACKGROUND

Energy-based surgical instruments are finding increasingly widespread applications in surgical procedures by virtue of their unique performance characteristics. Depending upon specific device configurations and operational parameters, energy-based surgical instruments can provide both transection of tissue and hemostasis of the tissue by coagulation, which may reduce or otherwise minimize patient trauma. Depending on the particular application, energy-based surgical instruments may utilize different surgical technologies including, for example, ultrasonic and/or electro-surgical (e.g., radio frequency (RF)) technologies.

A typical ultrasonic surgical instrument may include a handpiece containing an ultrasonic transducer and an elongated shaft assembly having a distally mounted end effector to effect the cutting and sealing of tissue. For example, the end effector may include a jaw assembly having an ultrasonic blade and a clamp arm, which may include a non-stick tissue pad or similar bed to receive the ultrasonic blade. In some cases, the elongated shaft assembly may be permanently affixed to the handpiece. In other cases, the elongated shaft assembly may be detachable from the handpiece, as in the case of a disposable shaft assembly or a shaft assembly that is interchangeable between different handpieces. In use, the end effector transmits ultrasonic energy to tissue brought into contact with the ultrasonic blade of the end effector to realize the cutting and sealing action. Such ultrasonic surgical devices may be configured for open surgical use, laparoscopic, and/or endoscopic surgical procedures including robotic-assisted procedures.

Ultrasonic energy cuts and coagulates tissue using temperatures lower than those used in electro-surgical procedures. Vibrating at high frequencies (e.g., 55,500 times per second), the ultrasonic blade denatures protein in the tissue to form a sticky coagulum. Pressure exerted on tissue by the ultrasonic blade surface collapses blood vessels and allows the coagulum to form a hemostatic seal. A surgeon can control the cutting speed and coagulation by the force applied to the tissue by the end effector, the time over which the force is applied, and the selected excursion level of the end effector.

In electro-surgical instruments, one or more electrodes are incorporated into the end effector and configured to apply therapeutic electrical current to the patient’s tissue to create a hemostatic seal. In electro-surgical instruments that do not include a harmonic mode (i.e., do not include a harmonic blade), the end effector may be embodied as two clamp arms or jaws. In such embodiments, the electro-surgical instrument may include a separate mechanical knife or blade for cutting the tissue after the creation of the hemostatic seal, which may be incorporated into the elongated shaft attached to the end effector. In bi-polar embodiments, an active electrode may be attached to one of the clamp arms of the end effector and configured to introduce an electrical current into the tissue, which is received by a return electrode attached to the other clamp arm of the end effector (or as the blade itself in embodiments including a harmonic mode). Conversely, in mono-polar embodiments, the return electrode (e.g., a “grounding pad”) may be separate from the electro-surgical instrument and located on a different part of the body of the patient. In some embodiments, the electro-surgical instrument may also be configured to apply a sub-therapeutic electrical current to the patient’s tissue, which may be used for sensing purposes (e.g., measuring tissue impedance).

Electro-surgery forms hemostatic seals by generating heat in the tissue via the introduced electrical energy, which is embodied as radio frequency (“RF”) energy. The particular frequency employed can vary based on the intended use of the electro-surgical instrument within the range of about 100kHz to 1 MHz, although higher frequencies can be employed in some embodiments. Additionally, sub-therapeutic frequencies may be used in some situations for purposes other than hemostatic sealing, such as performing various electrical measurements on the tissue.

It should be appreciated that some energy-based surgical instruments may employ dual or multi-modal technologies for the transection and/or hemostasis of patient tissue. For example, in some cases, an energy-based surgical instrument may include both ultrasonic and electro-surgical capabilities (e.g., by utilizing the ultrasonic blade as an electrode for the electro-surgery mode), which increases the surgical options provided by the surgical instrument to the surgeon.

### SUMMARY

According to an aspect of the present disclosure, a method for operating an energy-based surgical instrument may include determining, by a controller of the energy-based surgical instrument, whether application of an energy mode of the energy-based surgical instrument has been requested; controlling, by the controller and in response to a determination that the application of the energy mode has been requested, a jaw assembly of the energy-based surgical instrument to apply a pre-energy mode clamp pressure to patient tissue captured in the jaw assembly prior to application of the requested energy mode; activating, by the controller, the requested energy mode subsequent to application of the pre-energy mode clamp pressure to the patient tissue; and controlling, by the controller, the jaw assembly to apply an energy mode clamp pressure to the patient tissue while the requested energy mode is activated, wherein the energy mode clamp pressure is different from a pre-energy mode clamp pressure.

In some embodiments, controlling the jaw assembly of the energy-based surgical instrument to apply the pre-energy mode clamp pressure to patient tissue may include controlling, by the controller, the jaw assembly to apply a pre-energy mode clamp pressure to the patient tissue that increases over time. Additionally or alternatively, in some embodiments controlling the jaw assembly of the energy-based surgical instrument to apply the pre-energy mode clamp pressure to patient tissue may include controlling, by the controller, the jaw assembly to apply a pre-energy mode clamp pressure for a reference time period. For example, controlling the jaw assembly of the energy-based surgical instrument to apply the pre-energy mode clamp pressure to patient tissue may include controlling, by the controller, the jaw assembly to apply the pre-energy mode clamp pressure for a time period that is based on a measured compression of the patient tissue.

Additionally, in some embodiments, activating the requested energy mode subsequent to application of the pre-energy mode clamp pressure to the patient tissue may include delaying the application of the pre-energy mode after the application of the pre-energy mode clamp for a reference time period. In such embodiments, the method may further include controlling, by the controller, the jaw assembly to reduce the clamp pressure applied to the patient tissue from the pre-energy mode clamp pressure during the reference time period.

In some embodiments, the method may also include applying, by the controller, a sub-therapeutic radio frequency (RF) energy to the patient tissue to increase the temperature of the patient tissue to a reference temperature value prior to application of the requested energy mode. In some embodiments, the reference temperature is no greater than 70 degrees Celsius.

Additionally, in some embodiments, controlling the jaw assembly to apply the energy mode clamp pressure to the patient tissue while the requested energy mode is activated may include controlling, by the controller, the jaw assembly to apply an energy mode clamp pressure to the patient tissue based on an amount of energy applied to the patient tissue during the energy mode. Additionally or alternatively, in some embodiments, controlling the jaw assembly to apply the energy mode clamp pressure to the patient tissue while the requested energy mode is activated may include controlling, by the controller, the jaw assembly to apply an energy mode clamp pressure to the patient tissue that increases over time. Additionally or alternatively, in some embodiments, controlling the jaw assembly to apply the energy mode clamp pressure to the patient tissue while the requested energy mode is activated may include controlling, by the controller, the jaw assembly to apply an energy mode clamp pressure to the patient tissue based on a sensed characteristic of the patient tissue.

In some embodiments, the method may further include determining, by the controller, whether the application of the requested energy mode is completed; controlling, by the controller and in response to a determination that the application of the energy mode is completed, the jaw assembly of the energy-based surgical instrument to apply a pre-tissue transection clamp pressure to patient tissue prior to transection of the patient tissue; and transecting, by the energy-based surgical instrument, the patient tissue subsequent to application of the pre-tissue transection clamp pressure to the patient tissue.

In such embodiments, controlling the jaw assembly of the energy-based surgical instrument to apply the pre-tissue transection clamp pressure to patient tissue may include controlling, by the controller, the jaw assembly to apply a pre-tissue transection clamp pressure to the patient tissue for a reference time period. Additionally, in such embodiments, controlling the jaw assembly of the energy-based surgical instrument to apply the pre-tissue transection clamp pressure to patient tissue may include controlling, by the controller, the jaw assembly to apply a pre-tissue transection clamp pressure to the patient tissue that increases over time. Additionally or alternatively, in such embodiments, controlling the jaw assembly of the energy-based surgical instrument to apply the pre-tissue transection clamp pressure to patient tissue may include controlling, by the controller, the jaw assembly to apply a pre-tissue transection clamp pressure to the patient tissue based on an amount of energy applied to the patient tissue during the energy mode.

Additionally, in some embodiments, the energy mode of the energy-based surgical instrument may be a harmonic mode or a radio frequency (RF) mode. Furthermore, in some embodiments, the surgical instrument form part of a surgical robot. In such embodiments, the surgical instrument may be coupled to a robotic arm of the surgical robot, and the controller may be located separate from the surgical instrument.

According to another aspect of the present disclosure, an energy-based surgical instrument may include and end effector and a controller. The end effector may have a jaw assembly configured to grasp patient tissue and apply an amount of clamp pressure to the patient tissue. The controller may be configured to determine whether application of an energy mode of the energy-based surgical instrument has been requested; control, in response to a determination that the application of the energy mode has been requested, the jaw assembly to apply a pre-energy mode clamp pressure to the patient tissue prior to application of the requested energy mode; activate the requested energy mode subsequent to application of the pre-energy mode clamp pressure to the patient tissue; and control the jaw assembly to apply an energy mode clamp pressure to the patient tissue while the requested energy mode is activated, wherein the energy mode clamp pressure is different from a pre-energy mode clamp pressure.

In some embodiments, to activate the requested energy mode subsequent to application of the pre-energy mode clamp pressure to the patient tissue may include to delay the application of the pre-energy mode after the application of the pre-energy mode clamp for a reference time period. In such embodiments, the controller may be further configured to control the jaw assembly to reduce the clamp pressure applied to the patient tissue from the pre-energy mode clamp pressure during the reference time period.

Additionally, in some embodiments, the controller may be further configured to apply a sub-therapeutic radio frequency (RF) energy to the patient tissue to increase the temperature of the patient tissue to a reference temperature value prior to application of the requested energy mode. Additionally or alternatively, in some embodiments, the controller may be further configured to determine whether the application of the requested energy mode is completed; control, in response to a determination that the application of the energy mode is completed, the jaw assembly to apply a pre-tissue transection clamp pressure to patient tissue prior to transection of the patient tissue; and cause transection of the patient tissue subsequent to application of the pre-tissue transection clamp pressure to the patient tissue.

### BRIEF DESCRIPTION OF THE DRAWINGS

The detailed description particularly refers to the following figures, in which:

FIG. 1 is a simplified diagram of an embodiment of a system for performing an energy-based surgical procedure;

FIG. 2 is a perspective view of an embodiment of an energy-based surgical instrument of the system of FIG. 1;

FIG. 3 is a side elevation view of a jaw assembly of an end effector of the surgical instrument of FIG. 2 including an ultrasonic blade and in an open state;

FIG. 4 is a side elevation view of the jaw assembly of the end effector of the surgical instrument of FIG. 2 including an ultrasonic blade and in a closed state;

FIG. 5A is a perspective view of another embodiment of the end effector of the surgical instrument of FIG. 2 including an electrode on a lower jaw clamp of the jaw assembly;

FIG. 5B is a perspective view of another embodiment of the end effector of the surgical instrument of FIG. 2 including two jaw clamps, each having an electrode attached thereto;

FIG. 6 is an exploded view of the surgical instrument of FIG. 2;

FIG. 7 is a block diagram of a control circuit of the surgical instrument of FIG. 2;

FIG. 8 is a block diagram of another embodiment of a control circuit of the surgical instrument of FIG. 2 configured for operation based on time-dependent clamp pressure;

FIGS. 9 and 10 is a simplified flow diagram of a method for controlling an energy-based surgical instrument based on a time-dependent clamp pressure, which may be executed by the controller of FIG. 8;

FIG. 11 is an illustrative graph of tissue temperature correlated to clamp pressure graph over time during application of a radio frequency (RF) energy mode, which may be applied during the execution of the method of FIGS. 9 and 10;

FIG. 12 is an illustrative graph of tissue temperature correlated to clamp pressure graph over time during application of a harmonic energy mode, which may be applied during the execution of the method of FIGS. 9 and 10; and

FIG. 13 is a simplified block diagram of an embodiment of a robotic surgical system including a control console and a robotic manipulator, which may include the control circuit of FIG. 8.

### DETAILED DESCRIPTION OF THE DRAWINGS

While the concepts of the present disclosure are susceptible to various modifications and alternative forms, specific illustrative embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

Terms representing anatomical references, such as anterior, posterior, medial, lateral, superior, inferior, distal, proximal, et cetera, may be used throughout the specification in reference to the surgical instruments described herein as well as in reference to the patient’s natural anatomy. Such terms have well-understood meanings in both the study of anatomy and the field of surgery. Use of such anatomical reference terms in the written description and claims is intended to be consistent with their well-understood meanings unless noted otherwise.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

The disclosed embodiments may be implemented, in some cases, in hardware, firmware, software, or any combination thereof. The disclosed embodiments may also be implemented as instructions carried by or stored on a transitory or non-transitory machine-readable (e.g., computer-readable) storage medium, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

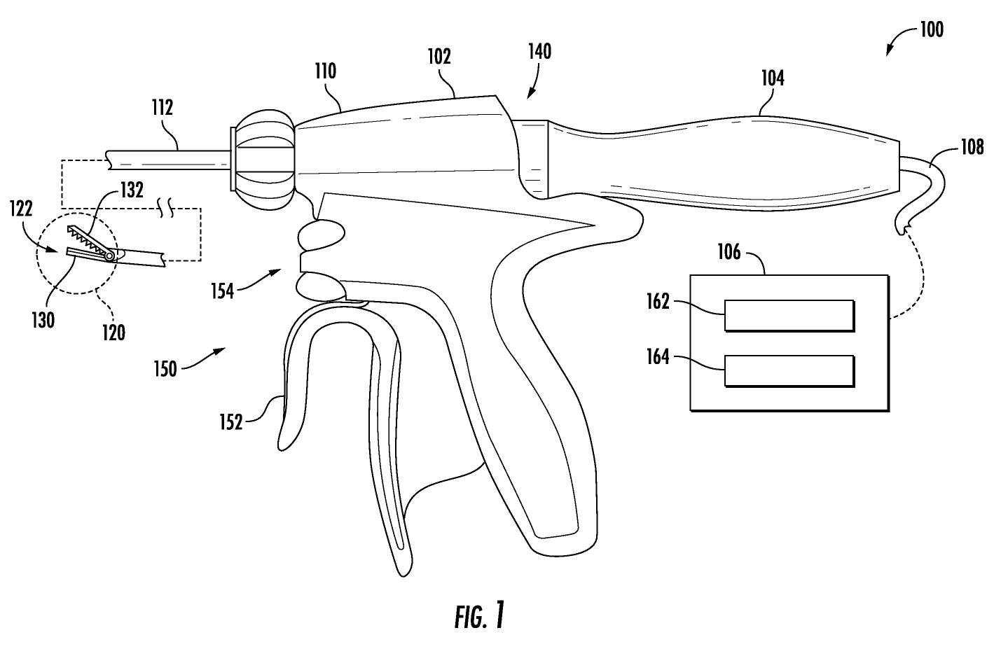

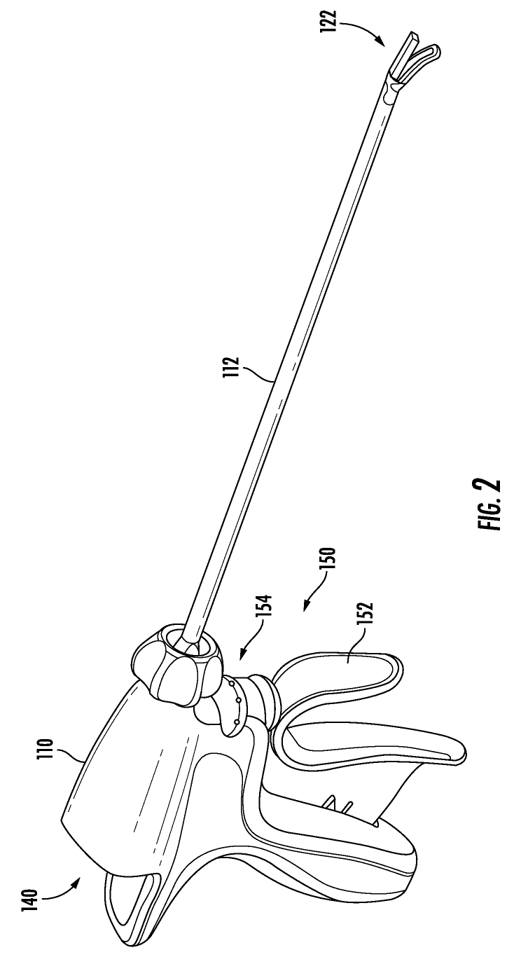

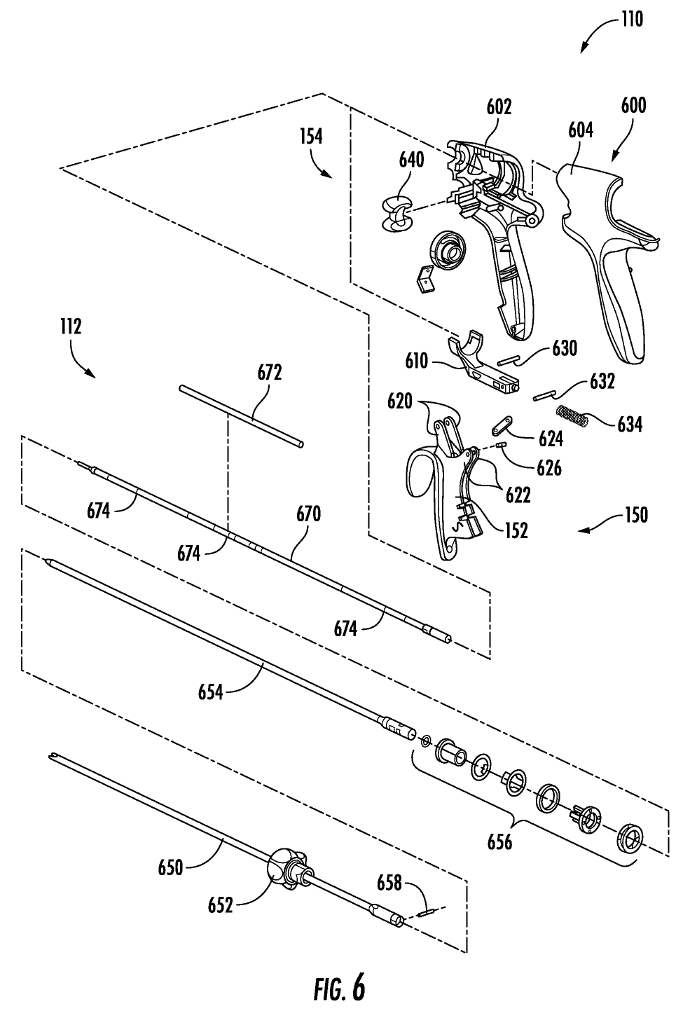

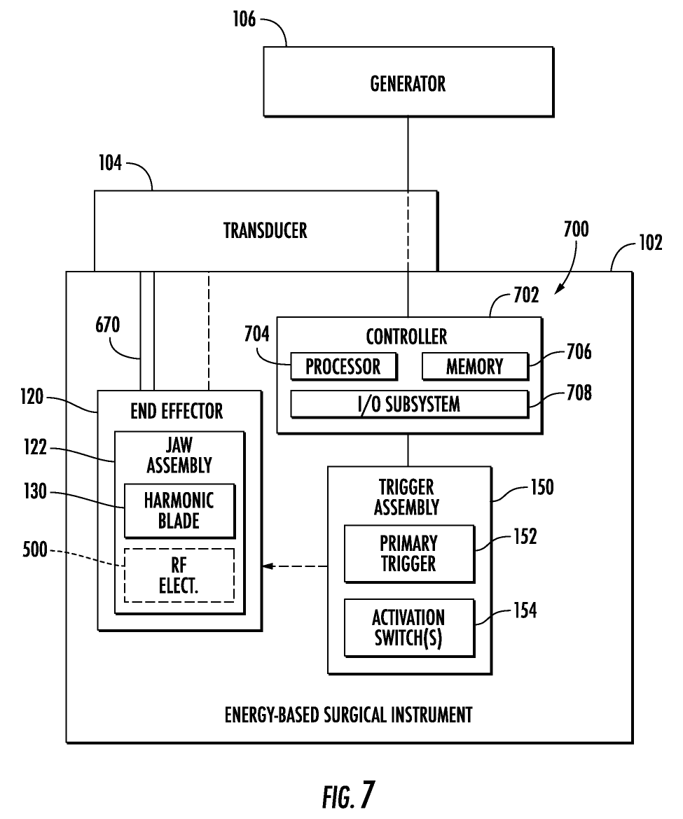

Referring now to FIGS. 1 and 2, in an illustrative embodiment, a system 100 for performing an energy-based surgical procedure includes a surgical instrument 102, a transducer 104, and a generator 106. The surgical instrument 102 is illustratively embodied as an ultrasonic surgical instrument, but may be embodied as an electro-surgical surgical instrument or a multi-modal, ultrasonic/elector-surgical surgical instrument in other embodiments. In use, the surgical instrument 102 is usable to perform various surgical procedures including laparoscopic, endoscopic, or traditional open surgical procedures. In doing so, a surgeon may selectively activate an ultrasonic mode (and/or an electro-surgical/RF mode) of the surgical instrument 102. In the ultrasonic mode, the generator 106 drives the transducer 104 to cause an ultrasonic blade 130 of a jaw assembly 122 of an end effector 120 of the surgical instrument 102 to vibrate at a reference frequency, which facilitates the contemporaneous cutting and hemostatic sealing of patient tissue. Additionally or alternatively, in some embodiments, the surgeon may selectively activate an electro-surgical mode of the surgical instrument 102 to deliver an amount of therapeutic RF energy to the patient tissue to effect hemostatic sealing. In such embodiments, the blade 130 may be embodied as an ultrasonic blade 130 or as a mechanical blade designed to cut tissue using mechanical force (e.g., in those embodiments not employing ultrasonic technologies). Furthermore, in some embodiments, the surgical instrument 102 may be configured with only an electro-surgical/RF mode and, in such embodiments, the jaw assembly 122 of the end effector 120 may not include the ultrasonic blade 130 as discussed in more detail below in regard to FIG. 5B.

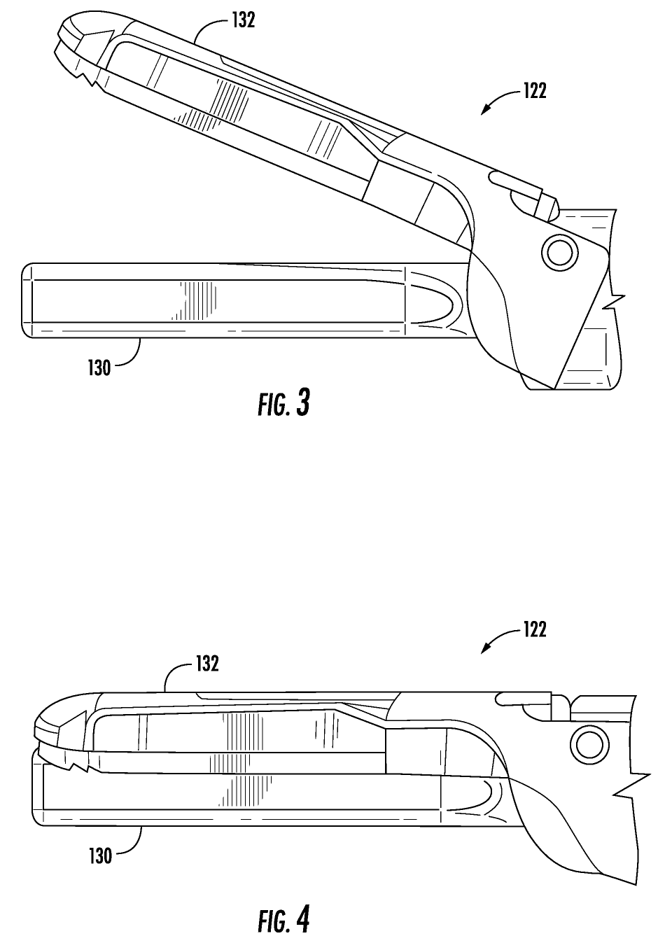

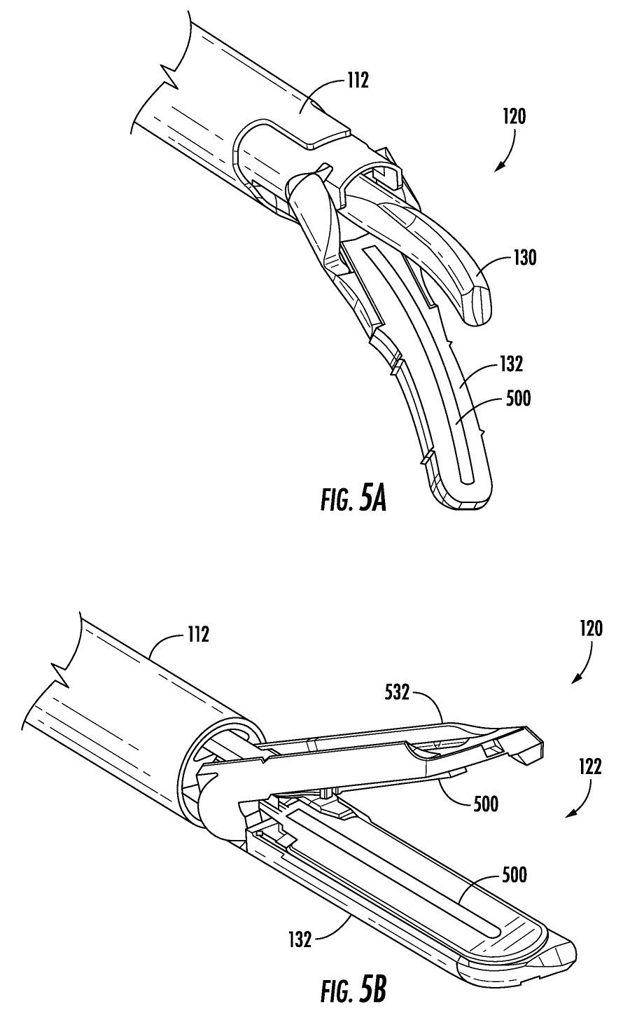

The surgical instrument 102 is illustratively embodied as ultrasonic surgical shears but may be embodied as other types of surgical instruments having an ultrasonic mode and/or electro-surgical mode in other embodiments. In the illustrative embodiment, the surgical instrument 102 includes a handle assembly 110 and an elongated shaft assembly 112, which extends distally away from the handle assembly 110 and may be removably attached to the handle assembly 110 in some embodiments. The elongated shaft assembly 112 includes the end effector 120 located at a distal end opposite the handle assembly 110. The end effector 120 includes the jaw assembly 122, which illustratively includes the ultrasonic blade 130 and a corresponding jaw clamp 132 (but may include two jaw clamps in those embodiments having only an electro-surgical/RF mode). As shown in FIGS. 3 and 4, the jaw assembly 122 is movable between an open state (FIG. 3) in which the jaw clamp 132 is positioned away from the ultrasonic blade 130 and a closed state (FIG. 4) in which the jaw clamp 132 is positioned near or otherwise contacts the ultrasonic blade 130. Actuation of the jaw assembly 122 from the open state to the closed state allows for the grasping, cutting, and coagulation of vessels and/or tissue by the jaw assembly 122. It should be appreciated that the open state may correspond to a degree of openness that is less than a fully opened position of the jaw assembly 122 and the closed state may correspond to a degree of closeness that is less than a fully closed position. That is, the closed state may, for example correspond to a minimal distance between the distal ends of the jaw clamp 132 and the ultrasonic blade 130 and the open state may correspond to a maximum distance between the distal ends of the jaw clamp 132 and the ultrasonic blade 130. However, in other embodiments, the open state may correspond to a fully opened position of the jaw assembly 122 and the closed state may correspond to a fully closed position of the jaw assembly 122.

In those embodiments in which the surgical instrument 102 includes both an ultrasonic mode and an electro-surgical/RF mode, the end effector 120 may include one or more RF electrodes 500 incorporated into the jaw clamp 132 as shown in FIG. 5A. Although the illustrative end effector 120 includes only a single electrode 500 in the embodiment of FIG. 5A, it should be appreciated that the end effector 120 may include additional electrodes 500 in other embodiments (e.g., multiple pads of electrodes 500). The electrode(s) 500 may be embodied as an active electrode configured to the RF energy or as a return electrode configured to “sink” an applied RF energy. In those embodiments utilizing bi-polar RF implementation, the ultrasonic blade 130 may embody the active or return electrode, with the electrode 500 embodying the other active or return electrode. Alternatively, other active or return electrodes may be incorporated on the ultrasonic blade 130 or in another part of the jaw assembly 122 of the end effector 120. In mono-polar implementation, the RF electrode(s) 500 may be embodied as an active electrode, and a return electrode may be attached to a portion of the patient’s body.

In those embodiments in which the surgical instrument 102 includes only an electro-surgical/RF mode, the jaw assembly 122 of the end effector 120 includes a jaw clamp 532 in place of the ultrasonic blade 130 as shown in FIG. 5B. In such embodiments, an electrode 500 may be attached to or otherwise incorporated into each jaw clamp 132, 532 and be embodied as an active or a return electrode to facilitate the application of RF energy to tissue captured between the jaw clamps 132, 532. In such embodiments, the surgical instrument 102 may include a knife incorporated into the elongated shaft assembly 112 that is configured to eject outwardly to cut the patient’s tissue after sealing of the tissue by the RF energy.

Referring back to FIGS. 1 and 2, in those embodiments including ultrasonic capabilities, the handle assembly 110 includes a receptacle 140 configured to receive the transducer 104 to facilitate connection of the transducer 104 to the handle assembly 110 and the elongated shaft assembly 112. The handle assembly 110 also includes a trigger assembly 150, which includes a primary trigger 152 and a switch assembly 154. The primary trigger 152 is operable by the surgeon to move the jaw assembly 122 of the end effector 120 between the open and closed states. The switch assembly 154 includes one or more buttons, which are selectable by the surgeon to activate (and configure, in some embodiments) the ultrasonic mode and/or the electro-surgical mode of the surgical instrument 102.

The transducer 104 is illustratively connected to the generator 106 by a cable assembly 108. As discussed above, the generator 106 is configured to drive the transducer 104 at a reference or resonant frequency to thereby cause the ultrasonic blade 130 to vibrate. For example, in an illustrative embodiment, the generator 106 may supply an electrical signal to the transducer 104 to cause the ultrasonic blade 130 of the jaw assembly 122 to vibrate longitudinally in the range of, for example, approximately 20 kHz to 250 kHz. In particular embodiments, for example, the ultrasonic blade 130 may vibrate in the range of about 54 kHz to 56 kHz (e.g., at about 55.5 kHz). In other embodiments, the ultrasonic blade 130 may vibrate at other frequencies including, for example, about 31 kHz or about 80 kHz. The excursion of the vibrations at the ultrasonic blade 130 can be controlled by, for example, controlling the amplitude of the electrical signal applied to the transducer 104 by the generator 106. The generator 106 may be activated so that electrical energy may be continuously or intermittently supplied to the transducer 104. The generator 106 also has a power line (not shown) for insertion in an electro-surgical unit or conventional electrical outlet. Additionally or alternatively, the generator 106 may be powered by a direct current (DC) source, such as a battery.

In some embodiments, the generator 106 may be configured to operate in different modes. In such embodiments, the generator 106 may include an ultrasonic generator module 162 for controlling an ultrasonic mode, an electro-surgical/Radio Frequency (RF) generator module 164 for controlling an electro-surgical mode, and/or other generator modules (e.g., a heat generator module) for controlling other operation modes. The various modes of the generator 106 may be operated independently of each other in some embodiments. For example, the generator 106 may activate the ultrasonic mode of the ultrasonic generator module 162 to apply ultrasonic energy to the jaw assembly 122 and subsequently, either therapeutic or sub-therapeutic RF energy may be applied to the jaw assembly 122 by the electro-surgical generator module 164. Alternatively, the activation modes of the generator 106 may be operated simultaneously or contemporaneously with each other.

In the electro-surgical mode, the electro-surgical generator module 164 is configured to generate RF energy at a frequency in the range of about 100 kilohertz (100 kHz) to about 1 megahertz (1 MHz). The generated RF energy is supplied to the patient’s tissue via the electrodes 500 of the end effector 120 as described above in regard to FIG. 5. In some embodiments, the electro-surgical generator module 164 may also be configured to selectively provide the RF energy at sub-therapeutic levels to perform various electrical measurements of the patient’s tissue. For example, the electro-surgical generator module 164 may be configured to measure an impedance of the patient’s tissue using the electrodes 500 and a suitable RF energy level.

Referring now to FIG. 6, as discussed above, the illustrative surgical instrument 102 includes the handle assembly 110 and the elongated shaft assembly 112, which extends distally away from the handle assembly 110. The handle assembly 110 includes a housing 600, which includes a right half-housing 602 and a left half-housing 604. The half-housings 602, 604 are configured to mate with each other to form the housing 600. To facilitate such mating, each of the half-housings 602, 604 may include various interfaces sized to mechanically align and engage one another to form the housing 600 and enclose the internal working components of the surgical instrument 102.

The primary trigger 152 of the trigger assembly 150 is coupled to a linkage mechanism to translate the rotational motion of the primary trigger 152 to axial motion of a yoke 610, which in turn is configured to move the jaw assembly 122 of the end effector 120 between the open and closed states via the elongated shaft assembly 112. The primary trigger 152 includes a first set of flanges 620 having openings formed therein to receive a first yoke pin 630, which extends through the yoke 610. The primary trigger 152 also includes a second set of flanges 622 configured to receive a first end of a link 624. A trigger pin 626 is received in openings formed in the first end of the link 624 and the second set of flanges 622. The trigger pin 626 forms a trigger pivot point for the primary trigger 152. A second end of the link 624, opposite the first end, is received in a slot formed in a proximal end of the yoke 610 and retained therein by a second yoke pin 632. As the primary trigger 152 is rotated about the pivot point formed from the trigger pin 626, the yoke 610 translates horizontally. A spring 634 is used to bias the yoke forward such that the jaw assembly 122 of the end effector 120 is biased to the open state (or a fully opened state).

As discussed above, the trigger assembly 150 also includes a switch assembly 154. The switch assembly 154 illustratively includes a toggle switch 640, which is selectable to activate one or more switches 642. Activation of the switches 642 electrically energizes an electrical element 644, which electrically energizes the ultrasonic transducer 104 to engage the ultrasonic mode of the surgical instrument 102.

The elongated shaft assembly 112 includes an outer tubular sheath 650 and a rotation knob 652 coupled to the outer cylindrical sheath 650. The rotation knob 652 is operable to rotate the outer cylindrical sheath 650 about an axis defined by the outer cylindrical sheath 650. A reciprocating tubular actuator 654 is located within the outer tubular sheath 650 and mechanically engaged with the end effector 120 on a distal end. The reciprocating tubular actuator 654 is also mechanically engaged, on a proximal end, with the yoke 610 within the handle assembly 110 via coupling elements 656. In embodiments including an ultrasonic mode, an ultrasonic waveguide 670 is located within the reciprocating tubular actuator 654. A distal end of the ultrasonic waveguide 670 is acoustically coupled (e.g., directly or indirectly mechanically coupled) to the ultrasonic blade 130, and a proximal end is acoustically coupled to the transducer 104. The ultrasonic waveguide 670 may be isolated from other components of the elongated shaft assembly 112 by a protective sheath 672 and a number of isolation elements 674. The outer tubular sheath 650, the reciprocating tubular actuator 654, and the ultrasonic waveguide 670 are mechanically engaged together via a pin 658.

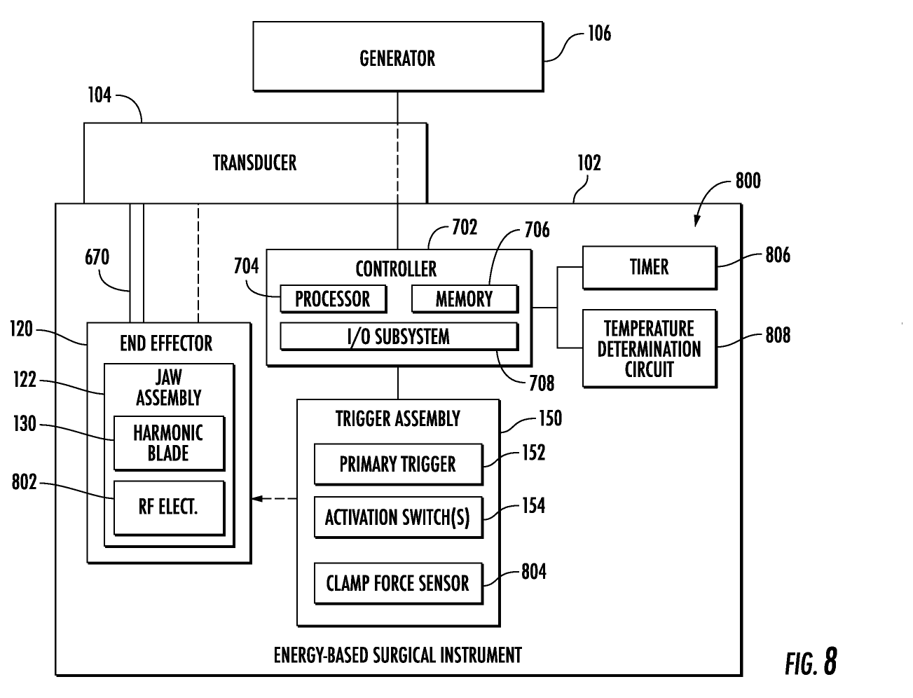

Referring now to FIG. 7, in the illustrative embodiment, the surgical instrument 102 includes a control circuit 700. The control circuit 700 includes a controller 702 and the trigger assembly 150, which cooperate to provide ultrasonic energy to the harmonic blade 130 of the jaw assembly 122 of the end effector 120 and/or RF energy to the RF electrodes 500 of the jaw assembly 122, depending on the operation modes of the surgical instrument 102 as discussed above. In other embodiments, however, the control circuit 700 may include additional or other electronic devices and/or circuit.

The controller 702 may be embodied as any type of controller, functional block, digital logic, or other component, device, circuitry, or collection thereof capable of performing the functions described herein. In illustrative embodiment, the controller 702 includes a processor 704, a memory 706, and an input/output (I/O) subsystem 708. The processor 704 may be embodied as any type of processor capable of performing the functions described herein. For example, the processor 704 may be embodied as a single or multi-core processor(s), digital signal processor, microcontroller, or other processor or processing/controlling circuit. Similarly, the memory 706 may be embodied as any type of volatile and/or non-volatile memory or data storage capable of performing the functions described herein. In operation, the memory 706 may store various data and software used during operation of the control circuit 700 such as executable firmware or software, programs, libraries, and drivers, which may be executed or otherwise used by the processor 704.

The processor 704 and memory 706 are communicatively coupled to other components of the control circuit 700 via the I/O subsystem 708, which may be embodied as circuitry and/or components to facilitate input/output operations between the controller 702 (e.g., the processor 704 and the memory 706) and the other components of the control circuit 700. For example, the I/O subsystem 708 may be embodied as, or otherwise include, memory controller hubs, input/output control hubs, firmware devices, communication links (i.e., point-to-point links, bus links, wires, cables, light guides, printed circuit board traces, etc.) and/or other components and subsystems to facilitate the input/output operations. In some embodiments, the I/O subsystem 708 may form a portion of a system-on-a-chip (SoC) and be incorporated, along with the processor 704 and the memory 706, and other components of the surgical instrument 102, on a single integrated circuit chip. Additionally, in some embodiments, the memory 706, or portions of the memory 706, may be incorporated into the processor 704.

During operation, as discussed above, the controller 702 is configured to control activation of an ultrasonic mode and/or an electro-surgical/RF mode of the surgical instrument 102. To do so, the controller 702 may monitor for activation of the primary trigger 152 and/or one or more activation switches 154 of the trigger assembly 150. In response to activation of the appropriate trigger 152 or switch 154, the controller 702 controls the transducer 104 to generate the ultrasonic energy, which is propagated to the harmonic blade 130 via the ultrasonic waveguide 670. Additionally or alternatively, in response to activation of a corresponding switch 154 of the trigger assembly 150, the controller 702 may be configured to supply an amount of RF energy, via the electro-surgical generator module 164 to the RF electrodes 500 via interconnections 710. It should be appreciated that, although the transducer 104 and the generator 106 are shown as separate components from the energy-based surgical instrument 102 in FIGS. 1 and 7, the transducer 104 and/or the generator 106 may be incorporated into the surgical instrument 102 in other embodiments.

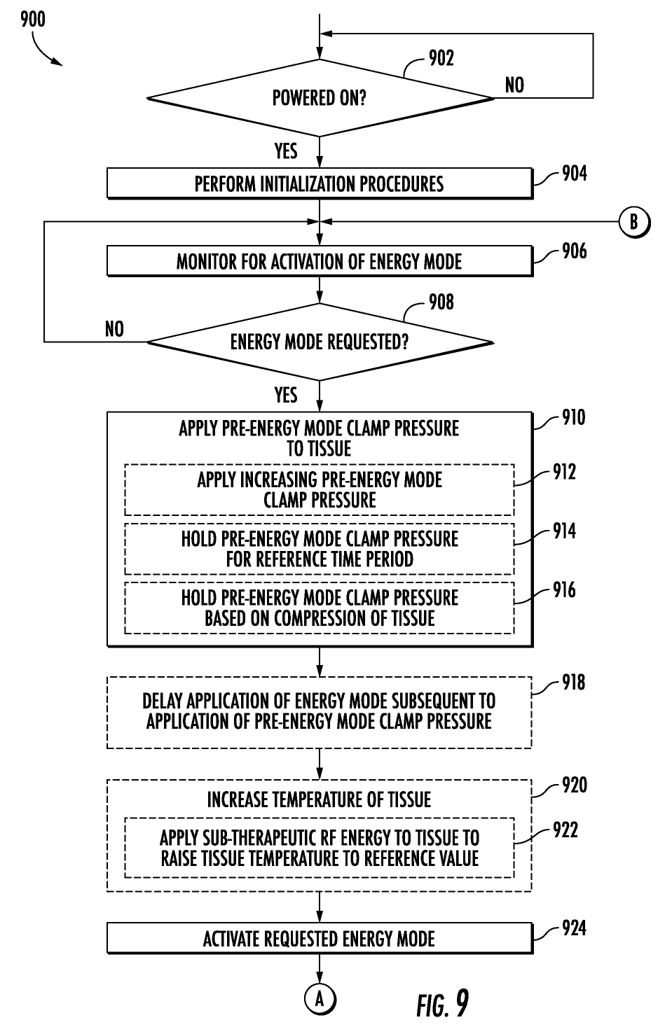

Referring now to FIG. 8, in another embodiment, the surgical instrument 102 is configured to control the jaw assembly 122 to apply or adjust clamp pressures to patient tissue captured in the jaw assembly 122 at different stages of the energy mode application. By applying different pressure over time to the tissue, relaxation of the tissue can be induced. Generally, tissue relaxation is proportionate to the magnitude of the pressure and the time over which that pressure is applied. Because higher pressure over time decreases the impedance of the tissue, the applied pressure levels and energy application time can drive power level changes required in the associated therapy.

As discussed in more detail below, the surgical instrument 102 is configured to apply a pre-energy mode clamp pressure to the patient tissue, an energy mode clamp pressure to the patient tissue, and pre-tissue transection clamp pressure to the patient tissue. The pre-energy mode clamp pressure is applied to the patient tissue prior to the application of a requested energy mode (e.g., a harmonic or RF mode). The energy mode clamp pressure is applied to the patient tissue during the application of the requested energy mode. And, the pre-tissue transection clamp pressure is applied to the patient tissue subsequent to the application of the energy mode and prior to transection of the patient tissue, which has undergone hemostasis via coagulation during the application of the requested energy mode. Additionally, in some embodiments, the surgical instrument 102 of FIG. 8 is configured to increase the temperature of the patient tissue prior to the application of the requested energy mode. For example, the tissue temperature of the patient tissue may be increased via application of an RF energy into the patient tissue during application of the pre-energy mode clamp pressure.

In such embodiments, the surgical instrument 102 may include a control circuit 800 as shown in FIG. 8. The control circuit 800 is substantially similar to the control circuit 700 described above and components of the control circuit 800 that correspond to similar components of control circuit 700 have been identified using the same reference number in FIG. 8 with the understanding that the description of such components provided above with regard to FIG. 7 applies to the corresponding components of the control circuit 800.

As shown in FIG. 8, the control circuit 800 includes a radio frequency (RF) electrode 802, a clamp force sensor 804, a timer 806, and a temperature determination circuit 808. The RF electrode 802 is included in the jaw assembly 122 of the end effector 120 and is substantially similar to the RF electrodes 500 described above. That is, the RF electrode 802 is usable to apply an amount of RF energy to patient tissue grasped by the jaw assembly 122. The RF electrode 802 may apply a therapeutic level of RF energy sufficient to, for example, create a hemostatic seal of the patient tissue and/or perform transection of the patient tissue. Additionally, the RF electrode 802 may apply a sub-therapeutic level of RF energy to perform various electrical measurements of the patient’s tissue, including sensing a temperature of the patient tissue.

The clamp force sensor 804 may be embodied as any type of sensor capable of generating sensor data indicative of an amount of clamp pressure asserted on the patient’s tissue by the jaw assembly 122. For example, the clamp force sensor 804 may be embodied as a piezoelectric pressure sensor, a strain gauge, or other type of sensor capable of generating sensor data indicative of the clamp pressure applied by the jaw assembly 122 or from which such clamp pressure can be determined. As discussed in more detail below, the illustrative energy-based surgical instrument 102 is configured to apply various amounts of clamp pressure to the captured patient tissue via the jaw assembly 122 at different stages of the energy mode application.

The timer 806 may be embodied as any type of timer circuit and associated components usable by the controller 702 to monitor a time period. For example, in some embodiments as described in more detail below, the controller 702 is configured to control the jaw assembly 122 to apply an amount of clamp pressure to the patient tissue for a reference time period, which is monitored and determined using the timer 806.

The temperature determination circuit 808 may be embodied as any type of electrical circuit and/or components configured to determine a temperature of the patient tissue captured in the jaw assembly 122. To do so, in some embodiments, the temperature determination circuit 808 may utilize the RF electrode 802 to monitor and determine the tissue temperature of the patient tissue. In some embodiments, as described in more detail below, the controller 702 may be configured to apply an amount of RF energy (e.g., via the RF electrode 802) to the patient tissue to increase the tissue temperature of the patient tissue prior to the application of a requested energy mode (e.g., to pre-condition the patient tissue for the energy mode).

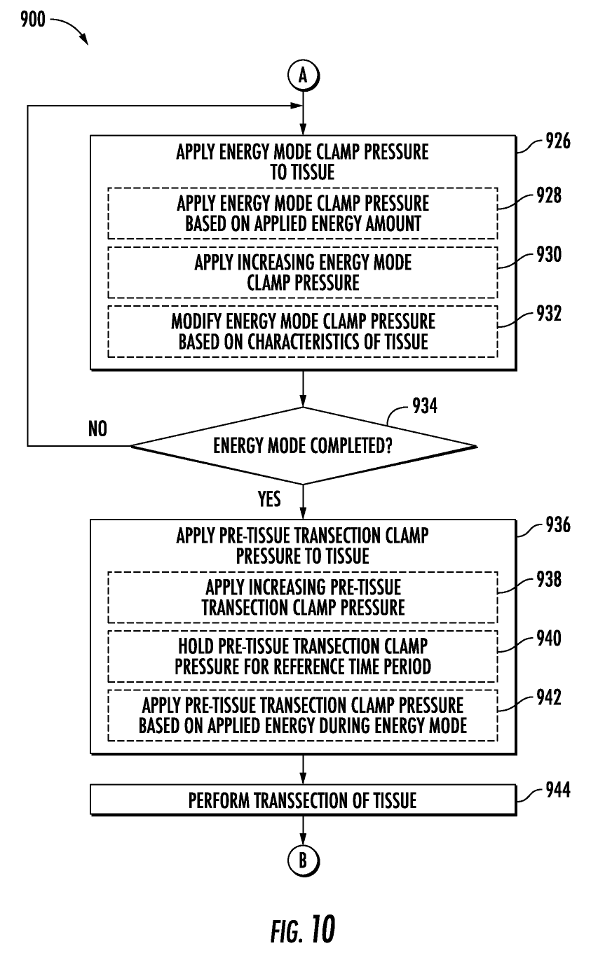

Referring now to FIG. 9, in use, the controller 702 of the energy-based surgical instrument 102 may execute a method 900 for controlling an energy-based surgical instrument based time-dependent clamp pressures. The method 900 begins with block 902 in which controller 702 determines whether the energy-based surgical instrument 102 has been powered to an on state. If so, the method 900 advances to block 904 in which the controller 702 performs one or more initialization procedures. For example, the controller 702 may verify operation of the jaw assembly 112, connection of the transducer 104, and/or other initialization or verification procedure.

After the controller 702 has performed the initialization procedures in block 904, the method 900 advances to block 906 in which the controller 702 monitors for a request for the activation of an energy mode of the energy-based surgical instrument 102. For example, the surgeon may request activation of an ultrasonic mode and/or an RF mode by operating a corresponding switch of the switch assembly 154 of the trigger assembly 150, as discussed above.

If the controller 702 detects a request for an energy mode of the energy-based surgical instrument 102 in block 908, the method 900 advances to block 910. In block 910, the controller 702 controls the jaw assembly 112 to apply a pre-energy mode clamp pressure to the patient tissue captured within the jaw assembly 112. That is, the controller 702 is configured to apply an amount of clamp pressure to the patient tissue prior to activating the requested energy mode (e.g., a ultrasonic and/or RF energy mode). It should be appreciated that by applying the pre-energy mode clamp pressure to the patient tissue, the patient tissue is allowed to creep to a more compacted form, which may mobilize liquid components out of the patient tissue and/or change the conductivity of the tissue, prior to application of the requested energy mode. In this way, the patient tissue may be preconditioned for the application of the requested energy mode.

The amount and duration of clamp pressure applied to the patient tissue during the pre-energy mode stage may be dependent on one or more factors. For example, in some embodiments in block 912, the controller 702 may control the jaw assembly 112 to apply a pre-energy mode clamp pressure to the patient tissue that increases over time. Additionally or alternatively, in block 914, the controller 702 may control the jaw assembly 112 to apply or hold the pre-energy mode clamp pressure for a reference time period. For example, in some embodiments, the controller 702 is configured to control the jaw assembly 112 to hold the pre-energy mode clamp pressure for a time period between 0.25 seconds to 1 second. In embodiments in which the pre-energy mode clamp pressure is increased, the clamp pressure may be held at various time points during the pressure increase and/or upon completion of the increase of the clamp pressure to a maximum pre-energy mode clamp pressure. Additionally, in some embodiments in block 916, the controller 702 may be configured to control the jaw assembly 112 to apply and/or hold the pre-energy mode clamp pressure based on compression of the patient tissue. That is, the controller 702 may be configured to sense a compression of the patient tissue using a suitable sensor and control the amount and duration of the application of the pre-energy mode clamp pressure based on the sensed compression of the patient tissue.

After the controller 702 has controlled the jaw assembly 112 to apply the pre-energy mode clamp pressure to the patient tissue in block 910, the controller 702 may be configured to delay the application of the requested energy mode in block 918. For example, in some embodiments, the controller 702 may delay the application of the requested energy mode for a time period between 3 seconds to 5 seconds. During the delay period time period, the controller 702 may maintain the pre-energy mode clamp pressure on the patient tissue or release the clamp pressure from the patient tissue to provide an amount of “rest” time for the patient tissue.

Additionally, in some embodiments, the controller 702 is configured to increase the temperature of the patient tissue in block 920 prior to application of the requested energy mode. To do so, in block 922, the controller 702 may apply sub-therapeutic radio frequency (RF) energy to the patient tissue to raise the tissue temperature to a reference value. To do so, as discussed above, the controller 702 may use the RF electrode 802 to apply the RF energy to increase the tissue temperature and monitor the temperature of the patient tissue.

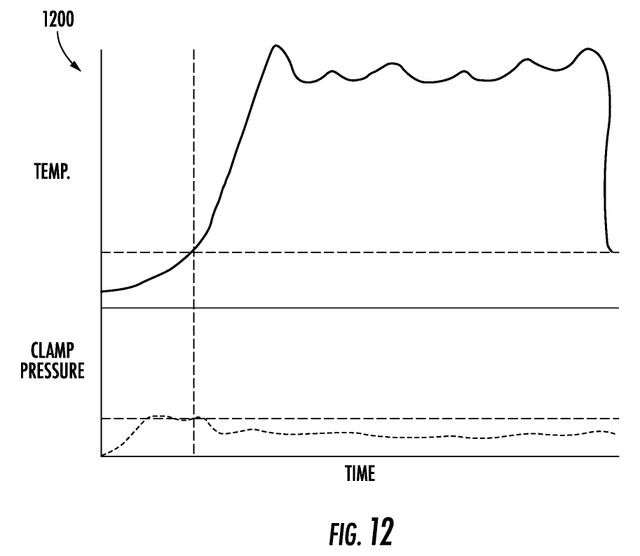

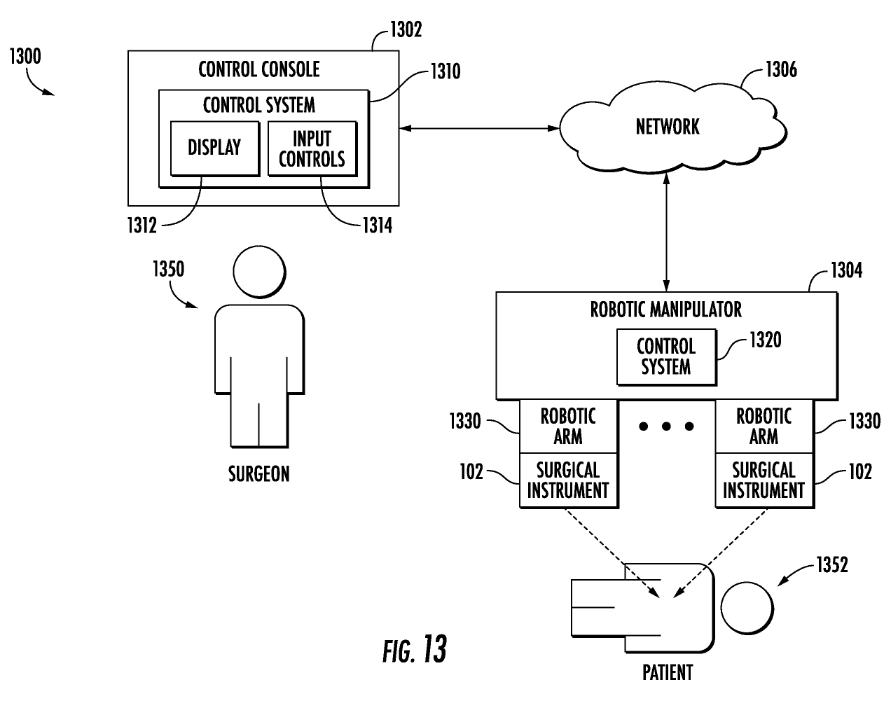

The controller 702 may be configured to control the temperature of the patient tissue subsequent to or during the application of the pre-energy mode clamp pressure. In this way, the controller 702 may apply or control a reference clamp pressure and tissue temperature to thereby precondition the patient tissue prior to application of the requested energy mode. For example, a graph 1100 of FIG. 11 illustrates a correlated tissue temperature and clamp pressure over time prior to, during, and subsequent to the application of a requested RF energy mode. Similarly, a graph 1200 of FIG. 12 illustrates a correlated tissue temperature and clamp pressure over time prior to, during, and subsequent to the application of a requested ultrasonic energy mode. As shown in each graph 1100 and 1200, each of the tissue temperature and the clamp pressure is increased to a reference level prior to activation of the corresponding energy mode.

Referring now back to FIG. 9, after the controller 702 has applied the pre-energy mode clamp pressure in block 910 and/or increased the tissue temperature in block 920, the method 900 advances to block 924 in which the controller 702 activates the requested energy mode. As discussed above, the requested energy mode may be an ultrasonic energy mode, an RF energy mode, or a combination of ultrasonic and RF energy modes.

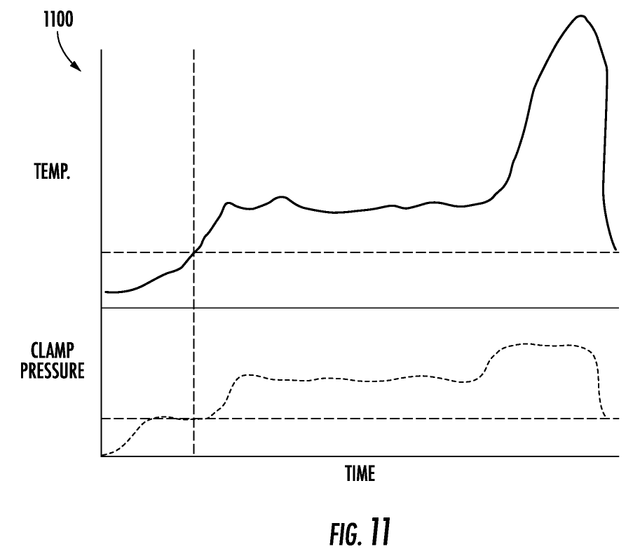

Regardless, while the requested energy mode is being applied to the patient tissue in block 924, the controller 702 controls the jaw assembly 112 to apply an energy mode clamp pressure to the patient tissue in block 926 of FIG. 10. That is, the controller 702 is configured to apply an amount of clamp pressure to the patient tissue during activation of the requested energy mode. It should be appreciated that by applying the energy mode clamp pressure to the patient tissue, the energy density applied by the energy mode may be amplified (e.g., by reducing the distance between the jaw assembly electrodes 802 and/or ultrasonic blade 130).

Similar to the pre-energy mode clamp pressure, the amount and duration of clamp pressure applied to the patient tissue during the energy mode stage may be dependent on one or more factors. For example, in some embodiments in block 928, the controller 702 may control the jaw assembly 112 to apply an energy mode clamp pressure to the patient tissue that is based on an amount of energy applied to the patient tissue during the requested energy mode. For example, during a requested RF energy mode, the energy mode clamp pressure may be based on an amount of RF energy applied to the patient tissue. In some embodiments, in block 930, the controller 702 may control the jaw assembly 112 to apply an energy mode clamp pressure to the patient tissue that increases over time. Additionally or alternatively, in block 932, the controller 702 may be configured to control the jaw assembly 112 to modify or adjust the energy mode clamp pressure based on sensed characteristics of the patient tissue. For example, in some embodiments, the controller 702 may control the jaw assembly 112 to reduce the amount of energy mode clamp pressure applied to the patient tissue based on a sensed compression of the patient tissue, based on a sensed tissue temperature, and/or other sensed characteristic of the patient tissue.

After the controller 702 has applied the energy mode clamp pressure to the patient tissue in block 926, the controller 702 determines whether the application of the energy mode has been completed in block 934. If not, the method 900 loops back to block 926 in which the controller continues to control the jaw assembly 112 to apply the energy mode clamp pressure to the patient tissue. If, however, the application of the requested energy mode is completed, the method 900 advances to block 936.

In block 936, the controller 702 controls the jaw assembly 112 to apply a pre-tissue transaction clamp pressure to the patient tissue. That is, the controller 702 is configured to apply an amount of clamp pressure to the patient tissue subsequent to the application of the requested energy mode and resulting hemostasis of the patient tissue and prior to transection of the patient tissue. For example, the pre-tissue transaction clamp pressure may be applied subsequent to an application of coagulation energy (i.e., the energy mode stage) and prior to application of a cut energy (i.e., the transection stage). It should be appreciated that by applying the pre-tissue transaction clamp pressure to the patient tissue, the patient tissue may be preconditioned for the subsequent transection procedure.

Similar to the pre-energy mode clamp pressure and the energy mode clamp pressure, the amount and duration of clamp pressure applied to the patient tissue prior tissue transection stage may be dependent on one or more factors. For example, in some embodiments in block 938, the controller 702 may control the jaw assembly 112 to apply a pre-tissue transaction clamp pressure to the patient tissue that increases over time. Additionally or alternatively, in block 940, the controller 702 may control the jaw assembly 112 to apply or hold the pre-tissue transaction clamp pressure for a reference time period. For example, in some embodiments, the controller 702 is configured to control the jaw assembly 112 to hold the pre-tissue transaction clamp pressure for a time period between 0.25 seconds to 1 second. In embodiments in which the pre-tissue transaction clamp pressure is increased, the clamp pressure may be held at various time points during the pressure increase and/or upon completion of the increase of the clamp pressure to a maximum pre-tissue transaction clamp pressure. Additionally, in some embodiments in block 942, the controller 702 may be configured to control the jaw assembly 112 to apply and/or hold the pre-tissue transaction clamp pressure based on an amount of RF energy applied to the patient tissue during the energy mode stage or an amount of RF energy applied to be applied to the patient tissue during the subsequent tissue transection stage.

Subsequently, in block 944, the controller 702 controls the energy-based surgical instrument 102 to perform the transection of the tissue. In this way, the controller 702 is configured to control the jaw assembly 112 to apply different clamp pressures to the patient tissue at different stages of energy mode stages. As discussed above, the applied clamp pressures may differ from each other and during the application to prepare or precondition the patient tissue for subsequent stages.

Although the above technologies of FIGS. 8-12 have been described in regard to the surgical instrument 102 illustrated in FIG. 8 including both a harmonic/ultrasonic mode and an electro-surgical/RF mode, the above-described technologies are also applicable to surgical instruments 102 having a single mode. For example, the method 900 may be implemented by a surgical instrument 100 including only a harmonic or ultrasonic mode. In such embodiments, control circuit 800 does not include the RF electrode(s) 802 and, as such, the method 900 may not include functionality of block 920 in which the temperature of the tissue is increased and/or other functionality reliant on the use of RF energy. Conversely, in other embodiments, the method 900 may be implemented by a surgical instrument 100 including only an electro-surgical or RF mode. In such embodiments, the control circuit 800 does not including the harmonic blade 130 and, as such, the transection of the tissue performed in block 944 may be performed using RF energy, rather than a harmonic blade, as described above.

Furthermore, in some embodiments, the surgical instrument 102 may be embodied as a robotic surgical instrument and form a portion of or otherwise be included in a robotic surgical system 1300 as shown in FIG. 13. The illustrative robotic surgical system 1300 includes a control console 1302 and a robotic manipulator 1304, which communicate with each other over a communication network 1306. Although only a single control console 1302 and a single robotic manipulator 1304 is shown in FIG. 13, it should be appreciated that the robotic surgical system 1300 may include additional control consoles 1302 and/or robotic manipulators 1304 in other embodiments.

The control console 1302 is usable by a surgeon 1350 to control the operation of the robotic manipulator 1304. To do so, the control console 1302 includes a control system 1310. Illustratively, the control system 1310 includes a display 1312 and one or more input controls 1314. However, it should be appreciated that the control system 1310 may include additional electrical components and devices, such as a processor, a memory, and a communication subsystem to enable communications of the components of the control system 1310, which are not illustrated in FIG. 13 for clarity.

The display 1312 may be embodied as any type of display device capable of generating images viewable by the surgeon 1350. In use, the display 1312 may display images related to the surgical procedure being performed via the robotic manipulator 1304. The displayed images may be obtained from, for example, an endoscopic camera operated by the robotic manipulator 1304. Additionally, the display 1312 may display information, including data determined by the control console 1302 and/or the robotic manipulator 1304, related to the surgical procedure (e.g., positional data of the robotic manipulator 1304).

The input controls 1314 are usable by the surgeon 1350 to control the functionality of the robotic manipulator 1304. The input controls 1314 may be embodied as any type of input device capable of receiving a corresponding input from the surgeon 1350. For example, the input controls 1314 may include physical controllers, such as joy sticks, hand-held actuator modules, exoskeletal gloves, and/or other input devices. The input controls 1314 may also include input devices other than hand-controlled devices such as foot pedals, vision tracking modules, and/or the like. In many embodiments, the input controls 1314 are movable in multiple degrees of freedom to control the positioning and operation of the robotic manipulator 1304.

The robotic manipulator 1304 also includes a control system 1320, which may be embodied as or otherwise include the control circuit 800 of FIG. 8, and one or more robotic arms 1330 to which surgical instruments 102 may be mounted. In some embodiments, the robotic manipulator 1304 may be mounted to a transport cart, sometimes referred to as an “arm cart,” that enables mobility of the robotic manipulator 1304 and the associated robotic arms 1330.

The robotic arms 1330 may include various articulable linkages and associated motors, which are controllable by the control console 1302 to move the corresponding robotic arm 1330 and any associated surgical instrument 840 to a desired position. For example, by manipulating an input control 1314 of the control console 1302, the surgeon 1350 may control the positioning of a corresponding robotic arm 1330, as well as the functionality of the associated surgical instrument 102.

In use, the robotic manipulator 1304 is positioned in close proximity to a patient 1352 requiring surgery. The robotic manipulator 1304 may be locked or mounted in place for the duration of the surgery. The surgeon 1350 may then manipulate the input controls 1314 to position one or more robotic arms 1330 and associated surgical instruments 1340 into a desired position. For example, the surgeon 1350 may position a robotic arm 1330 such that an associated surgical instrument 102 is inserted through a trocar or similar elongated passageway into the anatomical environment (e.g., the abdominal cavity of the patient 1352). Once so positioned, some surgical instruments 102 may be locked into position to avoid unintended repositioning.

The network 806 may be embodied as any type of wired and/or wireless network or set of communication links capable of facilitating communications between the control console 1302 and the robotic manipulator 1304. To do so, the network 1306 enable such communications using any suitable data communication specification and/or protocol. As such, in some embodiments, the network 1306 may include additional devices, such as additional computers, routers, stations, and/or switches, to facilitate such communications between the control console 1302 and the robotic manipulator 1304.

As discussed above, the control system 1320 is embodied as, or otherwise includes, the control circuit 800 of FIG. 8. As such, the control system 1320 is configured to execute the method 900 described above in regard to FIGS. 9 and 10. That is, the control circuit 800 is configured to control the jaw assembly 122 of the surgical instrument 102 to apply or adjust clamp pressures to patient tissue captured in the jaw assembly 122 at different stages of the energy mode application as discussed above in regard to method 900. Again, by applying different pressure over time to the tissue, relaxation of the tissue can be induced. Generally, tissue relaxation is proportionate to the magnitude of the pressure and the time over which that pressure is applied. Because higher pressure over time decreases the impedance of the tissue, the applied pressure levels and energy application time can drive power level changes required in the associated therapy as described above.

While the disclosure has been illustrated and described in detail in the drawings and foregoing description, such an illustration and description is to be considered as illustrative and not restrictive in character, it being understood that only illustrative embodiments have been shown and described and that all changes and modifications that come within the spirit of the disclosure are desired to be protected.

There are a plurality of advantages of the present disclosure arising from the various features of the methods, apparatuses, and systems described herein. It will be noted that alternative embodiments of the methods, apparatuses, and systems of the present disclosure may not include all of the features described yet still benefit from at least some of the advantages of such features. Those of ordinary skill in the art may readily devise their own implementations of the methods, apparatuses, and systems that incorporate one or more of the features of the present invention and fall within the spirit and scope of the present disclosure as defined by the appended claims.

1. A method for operating an energy-based surgical instrument, the method comprising:
determining, by a controller of the energy-based surgical instrument, whether application of an energy mode of the energy-based surgical instrument has been requested;
controlling, by the controller and in response to a determination that the application of the energy mode has been requested, a jaw assembly of the energy-based surgical instrument to apply a pre-energy mode clamp pressure to patient tissue captured in the jaw assembly prior to application of the requested energy mode;
activating, by the controller, the requested energy mode subsequent to application of the pre-energy mode clamp pressure to the patient tissue; and
controlling, by the controller, the jaw assembly to apply an energy mode clamp pressure to the patient tissue while the requested energy mode is activated, wherein the energy mode clamp pressure is different from a pre-energy mode clamp pressure.

2. The method of claim 1, wherein controlling the jaw assembly of the energy-based surgical instrument to apply the pre-energy mode clamp pressure to patient tissue comprises controlling, by the controller, the jaw assembly to apply a pre-energy mode clamp pressure to the patient tissue that increases over time.

3. The method of claim 1, wherein controlling the jaw assembly of the energy-based surgical instrument to apply the pre-energy mode clamp pressure to patient tissue comprises controlling, by the controller, the jaw assembly to apply a pre-energy mode clamp pressure for a reference time period.

4. The method of claim 3, wherein controlling the jaw assembly of the energy-based surgical instrument to apply the pre-energy mode clamp pressure to patient tissue comprises controlling, by the controller, the jaw assembly to apply the pre-energy mode clamp pressure for a time period that is based on a measured compression of the patient tissue.

5. The method of claim 1, wherein activating the requested energy mode subsequent to application of the pre-energy mode clamp pressure to the patient tissue comprises delaying the application of the pre-energy mode after the application of the pre-energy mode clamp for a reference time period.

6. The method of claim 5, further comprising controlling, by the controller, the jaw assembly to reduce the clamp pressure applied to the patient tissue from the pre-energy mode clamp pressure during the reference time period.

7. The method of claim 1, further comprising applying, by the controller, a sub-therapeutic radio frequency (RF) energy to the patient tissue to increase the temperature of the patient tissue to a reference temperature value prior to application of the requested energy mode.

8. The method of claim 7, wherein the reference temperature is no greater than 70 degrees Celsius.

9. The method of claim 1, wherein controlling the jaw assembly to apply the energy mode clamp pressure to the patient tissue while the requested energy mode is activated comprises controlling, by the controller, the jaw assembly to apply an energy mode clamp pressure to the patient tissue based on an amount of energy applied to the patient tissue during the energy mode.

10. The method of claim 1, wherein controlling the jaw assembly to apply the energy mode clamp pressure to the patient tissue while the requested energy mode is activated comprises controlling, by the controller, the jaw assembly to apply an energy mode clamp pressure to the patient tissue that increases over time.

11. The method of claim 1, wherein controlling the jaw assembly to apply the energy mode clamp pressure to the patient tissue while the requested energy mode is activated comprises controlling, by the controller, the jaw assembly to apply an energy mode clamp pressure to the patient tissue based on a sensed characteristic of the patient tissue.

12. The method of claim 1, further comprising:
determining, by the controller, whether the application of the requested energy mode is completed;
controlling, by the controller and in response to a determination that the application of the energy mode is completed, the jaw assembly of the energy-based surgical instrument to apply a pre-tissue transection clamp pressure to patient tissue prior to transection of the patient tissue; and
transecting, by the energy-based surgical instrument, the patient tissue subsequent to application of the pre-tissue transection clamp pressure to the patient tissue.

13. The method of claim 12, wherein controlling the jaw assembly of the energy-based surgical instrument to apply the pre-tissue transection clamp pressure to patient tissue comprises controlling, by the controller, the jaw assembly to apply a pre-tissue transection clamp pressure to the patient tissue for a reference time period.

14. The method of claim 12, wherein controlling the jaw assembly of the energy-based surgical instrument to apply the pre-tissue transection clamp pressure to patient tissue comprises controlling, by the controller, the jaw assembly to apply a pre-tissue transection clamp pressure to the patient tissue that increases over time.

15. The method of claim 12, wherein controlling the jaw assembly of the energy-based surgical instrument to apply the pre-tissue transection clamp pressure to patient tissue comprises controlling, by the controller, the jaw assembly to apply a pre-tissue transection clamp pressure to the patient tissue based on an amount of energy applied to the patient tissue during the energy mode.

16. An energy-based surgical instrument comprising:
an end effector having a jaw assembly configured to grasp patient tissue and apply an amount of clamp pressure to the patient tissue; and
a controller configured to:
determine whether application of an energy mode of the energy-based surgical instrument has been requested;
control, in response to a determination that the application of the energy mode has been requested, the jaw assembly to apply a pre-energy mode clamp pressure to the patient tissue prior to application of the requested energy mode;
activate the requested energy mode subsequent to application of the pre-energy mode clamp pressure to the patient tissue; and
control the jaw assembly to apply an energy mode clamp pressure to the patient tissue while the requested energy mode is activated, wherein the energy mode clamp pressure is different from a pre-energy mode clamp pressure.

17. The energy-based surgical instrument of claim 16, wherein to activate the requested energy mode subsequent to application of the pre-energy mode clamp pressure to the patient tissue comprises to delay the application of the pre-energy mode after the application of the pre-energy mode clamp for a reference time period.

18. The energy-based surgical instrument of claim 17, wherein the controller is further configured to control the jaw assembly to reduce the clamp pressure applied to the patient tissue from the pre-energy mode clamp pressure during the reference time period.

19. The energy-based surgical instrument of claim 16, wherein the controller is further configured to apply a sub-therapeutic radio frequency (RF) energy to the patient tissue to increase the temperature of the patient tissue to a reference temperature value prior to application of the requested energy mode.

20. The energy-based surgical instrument of claim 16, wherein the controller is further configured to:
determine whether the application of the requested energy mode is completed;
control, in response to a determination that the application of the energy mode is completed, the jaw assembly to apply a pre-tissue transection clamp pressure to patient tissue prior to transection of the patient tissue; and
cause transection of the patient tissue subsequent to application of the pre-tissue transection clamp pressure to the patient tissue.
